(19) United States
(12) Patent Application Publication
(10) Pub. No.: US 2021/0134402 A1
(43) Pub. Date: May 6, 2021

(54) METHODS AND SYSTEMS FOR PREDICTING SUCCESS RATES OF CLINICAL TRIALS

(71) Applicant: THE TRUSTEES OF COLUMBIA UNIVERSITY IN THE CITY OF NEW YORK, New York, NY (US)

(72) Inventors: Nicholas TATONETTI, New York, NY (US); Yun HAO, New York, NY (US)

(73) Assignee: THE TRUSTEES OF COLUMBIA UNIVERSITY IN THE CITY OF NEW YORK, New York, NY (US)

(21) Appl. No.: 17/085,688

(22) Filed: Oct. 30, 2020

Related U.S. Application Data
(63) Continuation of application No. PCT/US2019/036077, filed on Jun. 7, 2019.
(60) Provisional application No. 62/682,640, filed on Jun. 8, 2018.

(51) Int. Cl. G16H 10/20; G06K 9/62

(52) U.S. Cl. CPC G16H 10/20; G06K 9/6282; G06K 9/6277; G06K 9/6256

(57) ABSTRACT

System and methods for predicting success rates of clinical trials are disclosed. The system can comprise one or more processors and one or more computer-readable non-transitory storage media coupled to the one or more of processors including instructions operable when executed by one or more of the processor. The system is configured to cause the system to construct a training set using a data source, a performance score and a robustness score of the training set based on selected features, a random forest model based on the calculated performance and robustness scores; and calculate a toxicity score of the pharmaceuticals by applying the random forest model to a genome which is affected by the pharmaceuticals. Methods for predicting success rates of clinical trials and pharmaceuticals are also provided.

### CROSS REFERENCE TO RELATED APPLICATION

This application is a continuation of International Patent Application No. PCT/US2019/036077, filed Jun. 7, 2019, which claims the benefit of priority of U.S. Provisional Patent Application No. 62/682,640 filed Jun. 8, 2018, which are hereby incorporated by reference in their entireties.

### GRANT INFORMATION

This invention was made with government support under grant number R01-GM107145 awarded by the Nation Institutes of Health (NIH) and P30CA013696 awarded by National Cancer Institute (NCI). The government has certain rights in the invention.

### BACKGROUND

Assessing toxicity of therapeutic targets can be important in drug development. Drug toxicity can be a primary cause for attrition in drug development, accounting for 30% of certain clinical trial failures. In addition, drug toxicity can be a cause of hospital adverse events and injuries, affecting two million patients in the US annually. For instance, skin and gastrointestinal toxicity can be observed in patients receiving anti-EGFR therapy due to the indispensable role of EGFR activation in normal tissues. Similarly, hepatotoxicity of antiretroviral HIV therapy can be associated with the important function of target proteins such as purine nucleoside phosphorylase (PNP) and Pregnane X receptor (PXR) in the liver.

Certain methods using pharmacovigilance data to identify proteins associated with side effects do not consider tissue specificity. Other methods, including in silico quantitative structure-activity relationship (QSAR) models and in vitro screening of cell lines and organ-on-a-chip assays, can assess toxicity in a single tissue such as hepatotoxicity, nephrotoxicity, or cardiotoxicity. These methods can be costly and time-consuming and are often limited in their accuracy and translatability.

Accordingly, there remains a need to develop efficient and systematic techniques that connects targets to tissue toxicity.

### SUMMARY

The disclosed subject matter provides systems and methods for predicting success rates of pharmaceuticals and/or clinical trials. An example system can include one or more processors and one or more computer-readable non-transitory storage media coupled to one or more of the processors. The storage media can store instructions to cause the system to construct a training set using a data source, calculate a performance score and a robustness score of the training set based on selected features, select a random forest model based on the calculated performance and robustness scores; and calculate a toxicity score of the pharmaceuticals by applying the random forest model to a genome which can be affected by the pharmaceuticals. The performance score can be calculated based on a median Area Under Receive operating characteristic curve (AUROC). The median AUROC can be above 0.6. The robustness can be calculated based on absolute coefficients of two linear models. Higher score of the toxicity score can represent lower success rates of the pharmaceuticals. In non-limiting embodiments, the system can be further configured to validate the score based on clinical trial data using the pharmaceuticals. In some embodiments, the system can further improve an accuracy of the training set by dynamically adding additional clinical trial data.

In certain embodiments, the target feature can be a mRNA expression, a tolerance to genetic variation, an interaction with a cellular regulatory network, and/or a downstream pathway. In non-limiting embodiments, the pharmaceuticals can be a small molecule, a drug, a protein, a peptide, a virus, an enzyme, and/or a nucleic acid drug. In some embodiments, the data source can include a SNOMED, a SIDER, a DrugBank, and/or an Aggregate Analysis of Clinical Trials (AACT) database.

The disclosed subject matter also provides methods for predicting success rates of pharmaceuticals and/or clinical trials. An example method can include constructing a training set using a data source, calculating a performance score and a robustness score of the training set based on selected features, selecting a random forest model the calculated performance and robustness scores, and calculating a toxicity score of the pharmaceuticals by applying the random forest model to a genome which can be affected by the pharmaceuticals. In non-limiting embodiments, the method can further include validating the score based on clinical trial data using the pharmaceuticals. In some embodiments, the method can further include improving an accuracy of the training set by dynamically adding additional clinical trial data.

### BRIEF DESCRIPTION OF THE DRAWINGS

Further features and advantages of the present disclosure will become apparent from the following detailed description taken in conjunction with the accompanying figures showing illustrative embodiments of the present disclosure, in which:

FIG. 1 is a flow diagram illustrating a process of an exemplary system in accordance with the present disclosure.

FIGS. 2A-E are exemplary workflow and performance of the disclosed system in accordance with the present disclosure.

FIG. 3A is an illustration of TissueTox's performance using multiple types of features in accordance with the present disclosure. FIG. 3B is an illustration of TissueTox's robustness in accordance with the present disclosure. FIGS. 3C-3D are illustrations of distribution of receiver operating characteristic curves among 10 tissue models.

FIGS. 4A-4B are illustrations of predictive power of expression, variation, regulatory, and pathway figures in 10 tissue models.

FIG. 5 is an illustration of comparison of TissueTox scores across 17 protein classes. detection of image manipulation.

FIGS. 6A-6B are illustrations of comparison of TissueTox scores across ATC drug categories.

FIGS. 7A-7B are illustrations of comparison of TissueTox scores between targets associated with failed trials and targets associated with succeeded trials in 6 systems and 4 tissues. FIGS. 7C-7D are illustrations of comparison between drugs leading to the failure of trials and drugs leading the success of trials.

FIG. 8A is an illustration of ROC curves of four classifiers predicting the outcomes of clinical trials including structural-based method, a previously developed method named PrOCTOR, TissueTox scores-based method, and combining structural properties with TissueTox scores. FIG. 8B is an illustration of TissueTox scores-based models to 356 drugs currently undergoing clinical trials. FIG. 8C is an illustration of mRNA expression (upper) and predicted toxicity (lower) of mocetinostat targets across 45 GTEx tissues.

FIGS. 9A-9J are illustrations of performance comparison of TissueTox with other models in 10 body systems in accordance with the present disclosure.

FIG. 10A-10SS are illustrations of performance comparison of TissueTox with other models in 45 GTEx tissues in accordance with the present disclosure.

FIGS. 11A-11B are illustrations of comparison of TissueTox scores across 56 ATC drug categories.

FIG. 12 is an illustration of comparison of TissueTox scores between high- and low-confidence DILI-related targets in accordance with the present disclosure.

FIGS. 13A-13D are illustrations of comparison of TissueTox scores between failed and succeeded trials in 4 systems and 3 tissues in accordance with the present disclosure.

FIGS. 14A-14B are illustrations of predicted toxicity of trifluridine and pracinostat across 45 GTEx tissues in accordance with the present disclosure.

Throughout the figures, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments.

### DETAILED DESCRIPTION

The disclosed subject matter provides techniques for predicting success rates of clinical trials. The disclosed subject matter further provides techniques for assessing in vivo tissue toxicity of therapeutic targets. The disclosed systems and methods can use a target-based framework (i.e., TissueTox) which can assess the tissue-specific toxicity.

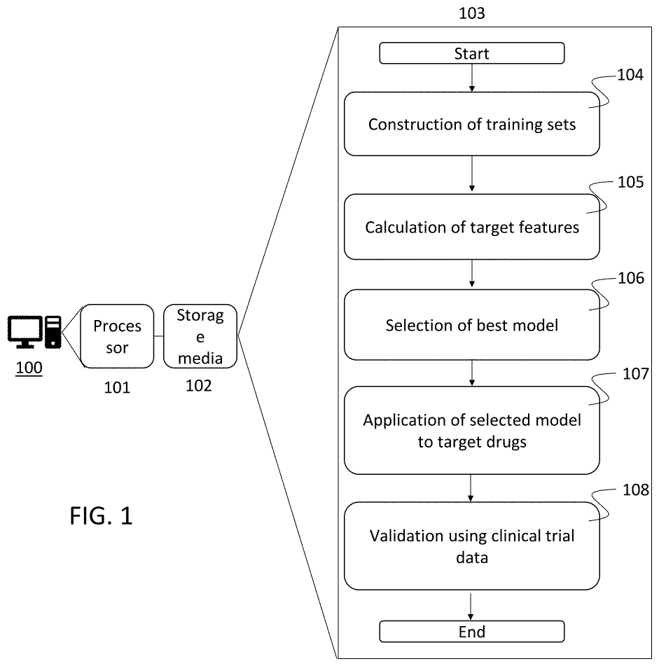

In certain embodiments, an exemplary system 100 can include one or more processors 101 and one or more computer-readable non-transitory storage media 102 coupled thereto. For example, the processor 101 can be an electronic circuitry (e.g., central processing unit, graphics processing unit, digital signal processor, etc.) within a computer/server 100 that can include a non-transitory storage media 102. Instructions 103 are a set of machine language that a processor can understand and execute. As shown in FIG. 1, the disclosed media 102 can include instructions 103 operable when executed by one or more of the processors 101 to cause the system 100 to perform various operations and analyses 104-108 for predicting success rate of clinical trials and assessing toxicity of therapeutic targets.

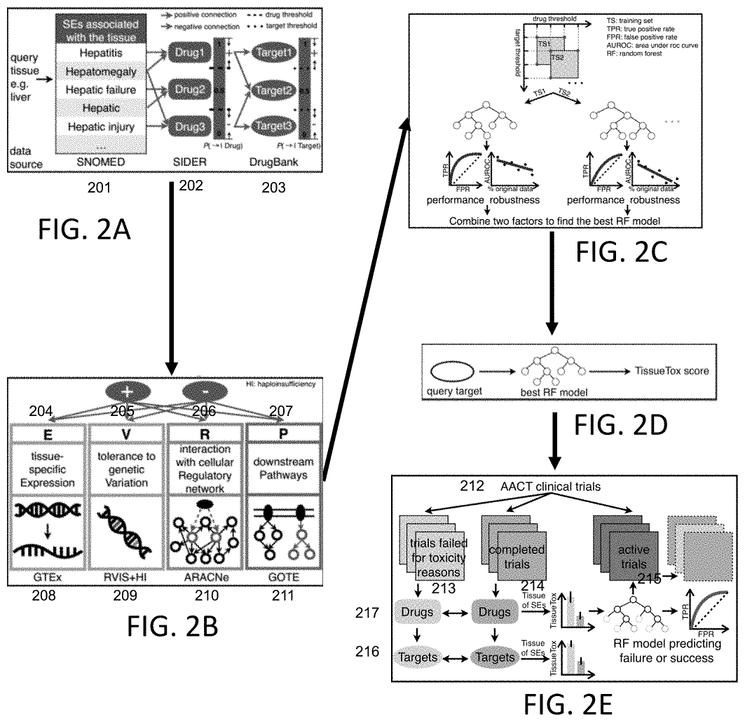

In certain embodiments, the disclosed system can be configured to construct a training set using a data source 104. The training set can be generated by integrating multiple data resources (e.g., SNOMED 201, SIDER 202, and DrugBank 203). For example, tissues can be connected to side effects using SNOMED 201, side effects can be connected to drugs using SIDER 202, and drugs can be connected to targets (e.g., proteins, genes, etc.) using DrugBank 203. Using data from the multiple data resources (e.g., drugs and side effects in human tissues and body systems), a reference dataset of targets and tissue toxicity can be established. A training set can be trained by the reference dataset for each of the systems and tissues. In non-limiting embodiments, to aggregate drugs across tissues and targets across drugs (which can have many to many relationships), thresholds (FIG. 2A, dashed lines) can be applied to reduce the number of spurious connections (e.g. off-target drug effects). For example, a filtering process can be used to reduce the mismatch between on-targets and off-target side effects. For each drug D, the probability of causing tissue toxicity (TT) P_{D→TT} can be calculated as:

$${P}_{D \to TT} = \frac{N ( related SEs caused by D )}{\begin{matrix}N ( related SEs caused by D ) + \\ N ( related SEs in which D is negative control )\end{matrix}} \qquad ( 1 )$$

where threshold T_{D} was used to define tissue toxicity of drugs as:

$${P}_{D \to TT} = \{ \begin{matrix}[ 0 , {T}_{D} ] & negative control \\ ( {T}_{D} , 1 - {T}_{D} ) & removed from the set \\ [ 1 - {T}_{D} , 1 ] & tissue factory\end{matrix} \qquad ( 2 )$$

For each target protein P, the probability of causing tissue toxicity P_{P→TT} can be calculated as

$${P}_{P \to TT} = \frac{N ( drugs targeting P that cause TT )}{\begin{matrix}N ( drugs targeting P that cause TT ) + \\ N ( drugs targeting P that do not cause TT )\end{matrix}} \qquad ( 3 )$$

The same method can be used to define tissue toxicity of target proteins with a threshold T_{P}. Different values (e.g., 0, 0.1, . . . , 0.4) can be applied to T_{D} and T_{P}. The value of T_{D} and T_{P} can be selected by calculation of target features, which can identify the training set with the least noise. FIG. 2A shows that five values for each of the thresholds can be applied resulting in 25 possible models.

In certain embodiments, the disclosed system can be configured to integrate multiple types of features to build random forest classifiers. For example, as shown in FIG. 2B, multi-omic features including mRNA expression (E, 204), tolerance to genetic variation (V, 205), interaction with cellular regulatory networks (R, 206), and/or pharmacological pathways (P, 207) can be incorporated into the TissueTox model.

In certain embodiments, the disclosed system can be configured to calculate performance and robustness of the model based on the integrated features 105 (FIG. 2C). The random forest model can be selected based on a balance between performance and robustness. For example, performance and robustness of the TissueTox model can be calculated based on mRNA expression, genetic variation, pharmacological pathway, and/or regulatory network. In non-limiting embodiments, the disclosed system can calculate at least two mRNA expression features per tissue, which can indicate the absolute and differential expression of a target (e.g., protein, gene, etc.) in the tissue. Absolute expression can be measured by the percentile of normalized mRNA expression data (RPKM) value among all genes. Differential expression can be measured by the absolute fold change derived from DESeq analysis. The DESeq can analyze count data from high-throughput sequencing assays such as RNA-sequencing for differential expression. In non-limiting embodiments, for each tissue type, the control samples can be generated using the following method. First, samples from other tissues of the same body system can be removed due to similarity in expression. Next, the remaining tissues can be averaged across replicates then grouped by the body system. For example, ten bootstrap samples can be drawn from each system to account for the imbalanced number of genotype-tissue expression (GTEx) tissues 208 from different systems. The bootstrap samples can be used as control for DESeq analysis.

The disclosed system can calculate variation features. For example, the disclosed system can calculate a Residual Variation Intolerance Score (RVIS) and a Haploinsufficiency (HI) score 205, which measure the tolerance of a target to genetic mutations. For example, to calculate the scores, number of common mutations that can affect gene function versus the number of all genetic variants per gene can be compared. Based on distribution of the common mutation and genetic variants, the RVIS can be estimated. If the RVIS<0, a gene can have fewer common functional mutations that expected (i.e., intolerance). If the RVIS>0, a given gene can have a comparatively high frequency of mutations that affect function (tolerance). Based on this score, all genes in the human genome can be ranked.

The disclosed system can calculate pharmacological pathway features. The disclosed system can use a data source (e.g., Reactome) for pathway analysis. To predict tissue-specific downstream pathways of targets, the disclosed system can include a program 207 (e.g., GOTE, MS-GOTE, DATE, and MS-DATE) which can manage multi-sample expression data sets such as GTEx. In GOTE, gene expression across tissues can be adjusted, and the distribution of all genes can be transformed into Gaussian to identify tissue-specific differential expressed genes (DEGs) based on deviation from the mean. In MS-GOTE, DESeq can be used to call DEGs as multiple samples of the same tissue. Bonferroni correction can be used to adjust the p-value and define DEGs as genes with adjusted p-value less than 0.05. Then, pathway enrichment analysis can be performed on the differentially expressed binding proteins of each transducer using Fisher's Exact Test, and the p-value can be transformed into Z-score. The Z-scores of each pathway derived from distinct transducers can be combined using Stouffer's Z-score method:

$${Z}_{combine} = \frac{{Σ}_{i ∈ transducers} {w}_{i} * {Z}_{i}}{\sqrt{{Σ}_{i ∈ transducers} {w}_{i}^{2}}} \qquad ( 4 )$$

where the Z-score of each transducer can be weighted by wᵢ. In GOTE, wᵢ can be defined as the expression of transducer Eᵢ. In MS-GOTE, the pearson correlation coefficient of RPKM across multiple samples Cᵢ can be calculated to measure the co-expression between the targets (e,g, GPCR) and transducer, which can be as an evidence to infer the coupling between them, with wᵢ set as the product of Eᵢ and Cᵢ. The combined Z-score was transformed to p-value, and pathways with p-value less than 0.05 were defined as downstream signaling pathways of the GPCR. MS-DATE can incorporate the results of DESeq analysis into DATE which can connect targets (e,g, non-GPCRs) to annotated pathways. In DATE, an expression Z-score can be calculated based on central limit theorem to assess the tissue-specific expression of genes in a pathway, then a non-GPCR can be connected to an annotated pathway when the Z-score is greater than 1.64. In MS-DATE, the tissue-specific expression can be assessed by testing whether the pathways genes are enriched among DEGs using Fisher's Exact Test, and a non-GPCR can be connected to an annotated pathway when the p-value is less than 0.05. In non-limiting embodiments, pathways with less than 5 or more than 100 annotated proteins can be analyzed by the disclosed system. To reduce the redundancy among predicted pathways, the hierarchy of Reactome can be used to filter out pathways that were connected to a target along with their descendants. Each predicted pathway can be regarded as a binary feature in the TissueTox model, which can indicate whether the pathway can be connected to a target or not.

In certain embodiments, the disclosed system can calculate at least two regulatory features per tissue. For example, a recall feature and a precision feature can be calculated by measuring the efficacy of targets to modify the activity of master regulators through downstream pathways (DPs). The disclosed system can include an analysis tool (e.g., ARACNe 206) to infer tissue-specific gene regulatory network from normalized mRNA expression data (RPKM) of each GTEx tissue. In non-limiting embodiments, VIPER can be used to infer the activity of transcription factors (TFs) regulating gene expression. TFs with certain activity (P<0.05) can be defined as master regulators (MRs). Recall was defined as the weighted proportion of MRs that can be regulated by the DPs of a target while precision can be defined as the weighted proportion of DPs that effectively regulate MRs:

$$\begin{matrix}Recall = \frac{{Σ}_{i ∈ MRs} - log {P}_{i} * I ( i annotated in DPs )}{{Σ}_{i ∈ MRs} - log {P}_{i}} & ( 5 ) \\ Precision = \frac{{Σ}_{j ∈ DPs} \frac{- log {P}_{j}}{length ( j )} * I ( j contains MRs )}{{Σ}_{j ∈ DPs} \frac{- log {P}_{j}}{length ( j )}} & ( 6 )\end{matrix}$$

where I is the indicator function, MRs are weighted by the p-value derived from VIPER analysis Pᵢ, and DPs are weighted by the ratio of p-value derived from the pathway analysis Pⱼ versus the number of proteins in the pathway.

The term “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 20%, preferably up to 10%, more preferably up to 5%, and more preferably still up to 1% of a given value. Also, particularly with respect to systems or processes, the term can mean within an order of magnitude, preferably within five-fold, and more preferably within two-fold, of a value.

In certain embodiments, the disclosed system can train and select a TissueTox model based on the integrated features 106. For example, using the features above (e.g., mRNA expression, variation, pathway, and regulatory features), about 100 random forest classifiers with about 500 trees each can be built for every training set derived for a tissue/system. Results can be averaged over the 100 classifiers to account for the stochastic nature of random forest. The out-of-bag probability can be used to evaluate the performance of each model, which can be measured by the AUROC (FIG. 2C). To prevent over-fitting, the disclosed system can randomly remove 10, 20, . . . , 50 percent samples or features from each training set and recalculate the AUROC of new models. The removal can be repeated about 100 times to account for the stochastic nature of sampling. In non-limiting embodiments, two linear regression models can fit using the normalized AUROC against the percentage of samples and features left to rebuild the model. The model robustness can be measured by the absolute coefficients of two linear models: ksample and kfeature. The performance and robustness scores can be normalized across all models derived for the same tissue/system using median absolute deviation (MAD) modified Z-scores, which can be combined using Stouffer's method. Specifically,

$$\begin{matrix}Modified {Z}_{i} = \frac{0.6745 ( {x}_{i} - \overline{x} )}{MAD} & ( 7 ) \\ Combined Z = \frac{{w}_{AUROC} * {Z}_{AUROC} + {w}_{{k}_{sample}} * {Z}_{{k}_{sample}} + {w}_{{k}_{feature}} * {Z}_{{k}_{feature}}}{\sqrt{{w}_{AUROC}^{2} + {w}_{{k}_{sample}}^{2} + {w}_{{k}_{feature}}^{2}}} & ( 8 )\end{matrix}$$

where w_{AUROC}, wₖ_{ₛₐₘₚₗₑ}, wₖ_{_{feature}} can be the weights used to combine three measurements and can be set as 1, 0.5, 0.5 to ensure that performance and robustness were equally considered in model selection. The model with the highest combined Z-score can be selected for each tissue/system. In some embodiments, an importance score of each feature can be measured by the increase in mean squared error (MSE) when the feature is removed from the model. The importance score can be then normalized by the sum across all features in each model. In non-limiting embodiments, a True Positive Rate (i.e., proportion of predicted that are true) and a False Positive Rate (i.e., proportion of predicted that are false) of training sets can be assessed to determine the performance and robustness scores.

In certain embodiments, the disclosed system can apply the selected model of each tissue/system to the human druggable genome 107. For example, the human druggable genome can be curated by integrating databases (e.g., dGene, GtoPdb, and DrugBank). Druggable proteins can be classified into major classes (e.g., GPCRs, nuclear hormone receptors, ion channels, transporters, catalytic receptors, enzymes, and other proteins). Then, the selected random forest model of each tissue/system can be applied to calculate the probability of causing tissue toxicity, which can be defined as the TissueTox score (FIG. 2D). Proteins/pharmaceuticals with TissueTox scores higher than the median of druggable genome in all body systems can be defined as Toxic proteins/pharmaceuticals. In non-limiting embodiments, the pharmaceuticals can include a small molecule, a drug, a protein, a peptide, a virus, an enzyme, and/or a nucleic acid drugs.

In certain embodiments, the disclosed system can validate the TissueTox score by using clinical trial data 108. For example, as shown in FIG. 2E, curated data of clinical trials can be obtained from a database (e.g., AACT 212). Failed trials 213 for toxicity reasons can be extracted, and multiple trials 214 can be extracted as negative controls. The failed trials 213 can be identified by overall status of “terminated”, “suspended”, or “withdrawn”, along with specified toxicity or safety reasons that led to the failure. The control trials 214 can be identified by overall status of “completed”. Data regarding drugs administrated in each clinical trial and their observed side effects can be extracted from the database. Target proteins of the drugs can be also obtained from a database (e,g., DrugBank). To ensure that the validation is independent of the model construction, the drugs or target proteins can be removed from the training sets of TissueTox models if they appears in the dataset (e.g., AACT 212), then the models can be rebuilt with the rest of training data regenerating TissueTox scores of all proteins in human druggable genome. TissueTox scores can be compared on at least two levels: target proteins 216 and drugs 217. TissueTox score of a drug can be defined as the average scores of target proteins for actual clinical trials 215.

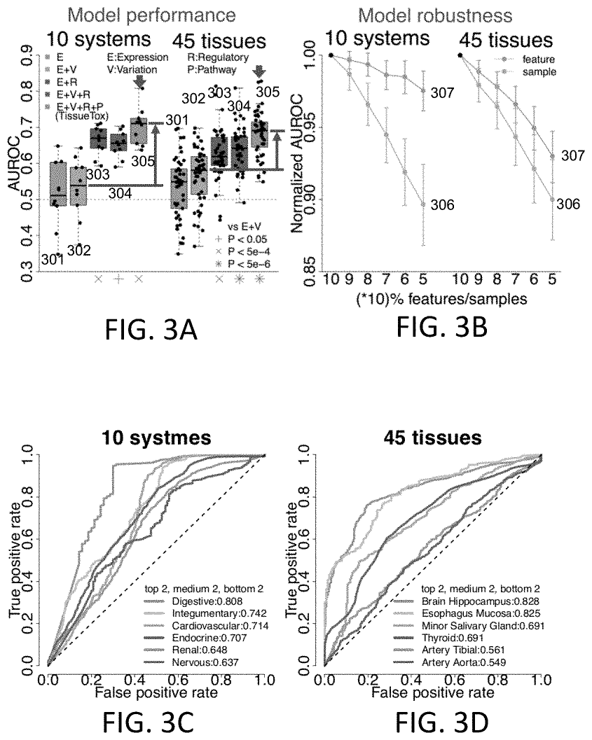

As shown in FIGS. 3A-3D, the performance of the disclosed system can be improved by adding the regulatory and pathway features in the model. FIG. 3A shows performance of TissueTox as well as other models built using one, two, or three types of features (i.e., E 301, E+V 302, E+R 303, E+V+R 304, and E+V+R+P 305). The performance can be measured by the area under receiver operating characteristic curve (AUROC) of each model. Significance assessed using one-sided T test. FIG. 3B shows robustness of TissueTox, which can be measured by the change in AUROC when using partial samples 306 or features 307 to rebuild the model. Results can be averaged across 10 system models and 45 tissue models with 95% confidence interval. FIGS. 3C and 3D show the distribution of receiver operating characteristic (ROC) curves among 10 tissue models (3C) and 45 system models (3D). Six models with the top, medium, and bottom two ranked AUROC values can be plotted. FIGS. 3C-3D show that the median area under receiver operating characteristic curve (AUROC) can be 0.711 (95% CI: 0.652-0.729) across the 10 systems and 0.691 (95% CI: 0.671-0.704) across the 45 tissues. In non-limiting embodiments, the performance of the disclosed system can be remained robust against the partial removal of features or samples. Robustness of TissueTox, which was measured by the change in AUROC when using partial samples or features to rebuild the model. For example, about 90% of original AUROC can be retained with 50% of the data (FIG. 3B), suggesting that TissueTox models can avoid overfitting the training data. In some embodiments, pathway features can improve the predictive power.

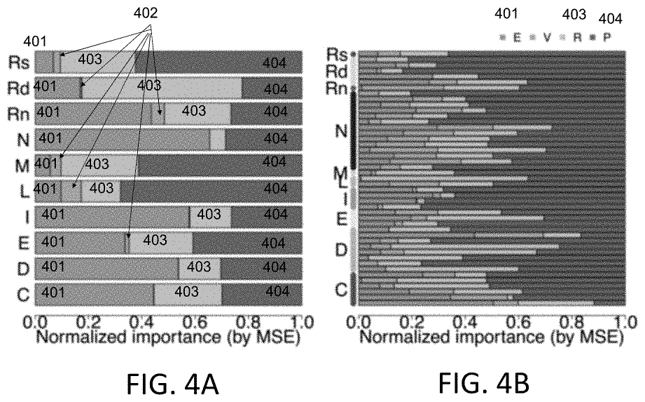

The disclosed system with the pathway features integrated can show about 40±10% of the normalized importance among 10 systems (FIG. 4A) and 53±5% among 45 tissues (FIG. 4B). FIGS. 4A-4B shows the predictive power of expression 401, variation 402, regulatory 403, and pathway 404 features in 10 tissue models and 45 system models, which can be measured by a normalized importance score proportional to the increase in mean squared error (MSE) when the feature are removed from the model. The normalized importance scores of four types can be shown as stacked bars for each model (in an order of E, V, R and P). All 45 tissues were grouped by the 10 systems on y-axis in (FIG. 4B). Certain features can show different predictive power based on the level of targets. For example, as shown in FIG. 4B. expression features can show higher predictive power in systems (34±14%) compared to tissues (14±3%).

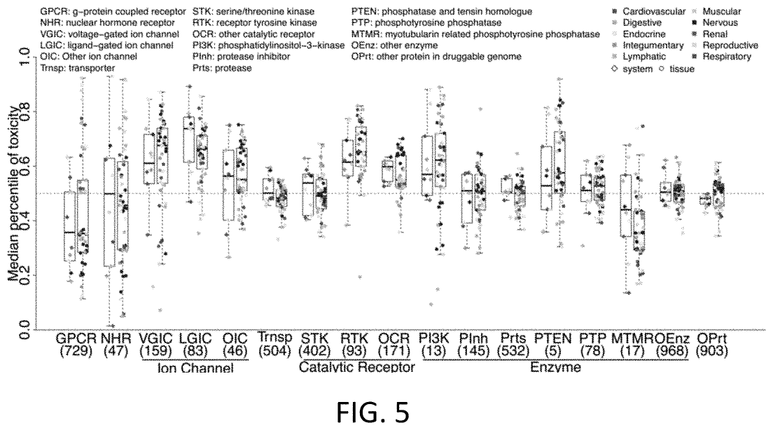

In certain embodiments, the disclosed system can predict TissueTox scores across protein classes and provide distinct levels of toxicity as well as tissue-specificity within each class. For example, FIG. 5 shows TissueTox scores of 4,857 proteins in the human druggable genome across 17 protein classes. GPCRs can be predicted with low toxicity in most systems except reproductive system while ion channels can be predicted with high toxicity in the nervous system due to their high expression in these tissues. NHRs show high variability of predicted toxicity across systems, ranging from low toxicity in the renal system to high toxicity in the reproductive system, while transporters and proteases average toxicity consistently across systems. Certain targets of cancer therapy such as RTKs, STKs, PI3Ks, and PTEN can exhibit high predicted toxicity in the digestive or integumentary system, where most side effects can be observed among patients receiving the therapy. Ion channels can be toxic to nervous system. The median percentile scores are shown as boxplot with jitter points for 10 systems (diamond) and 45 tissues (circle).

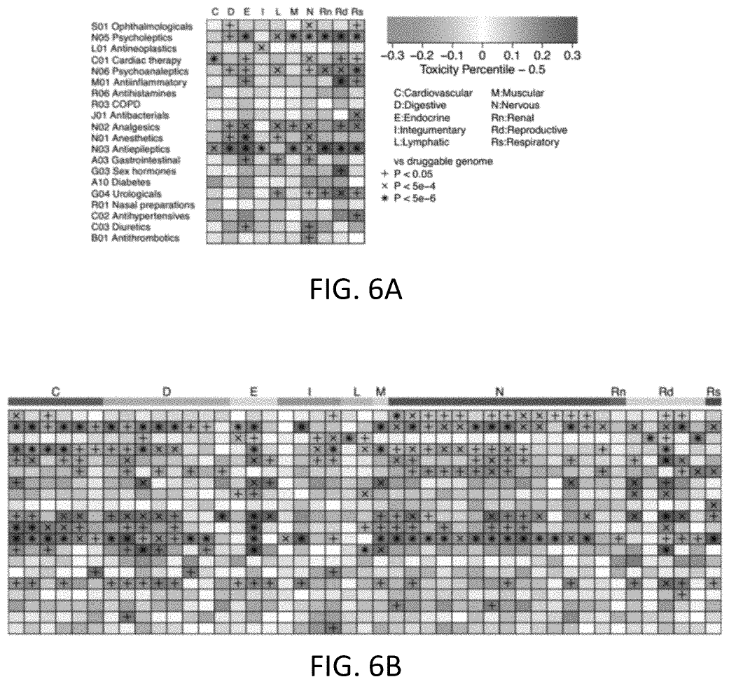

In non-limiting embodiments, the prediction of the disclosed system can identify the tissue-specific toxicity of several categories (e.g., antineoplastics in integumentary system and antibacterials in respiratory system). FIGS. 6A and 6B show comparison of TissueTox scores across ATC drug categories. The results of 20 categories with the highest number of drugs can be shown. The ATC code of each category is shown on the left along with annotation. The toxicity of each category can be measured by the average percentile of TissueTox scores among all 4,857 proteins. The average percentile scores are shown as two heatmaps for 10 systems (6A) and 45 tissues (6B). All 45 tissues are grouped by the 10 systems on x-axis in (6B). The significance levels of two-sided T test against all 4,857 proteins were shown in the cells with adjusted p-value less than 0.05. In-non-limiting embodiments, the disclosed system also can identify connections between targets and drug-induced injury (e.g., liver injury).

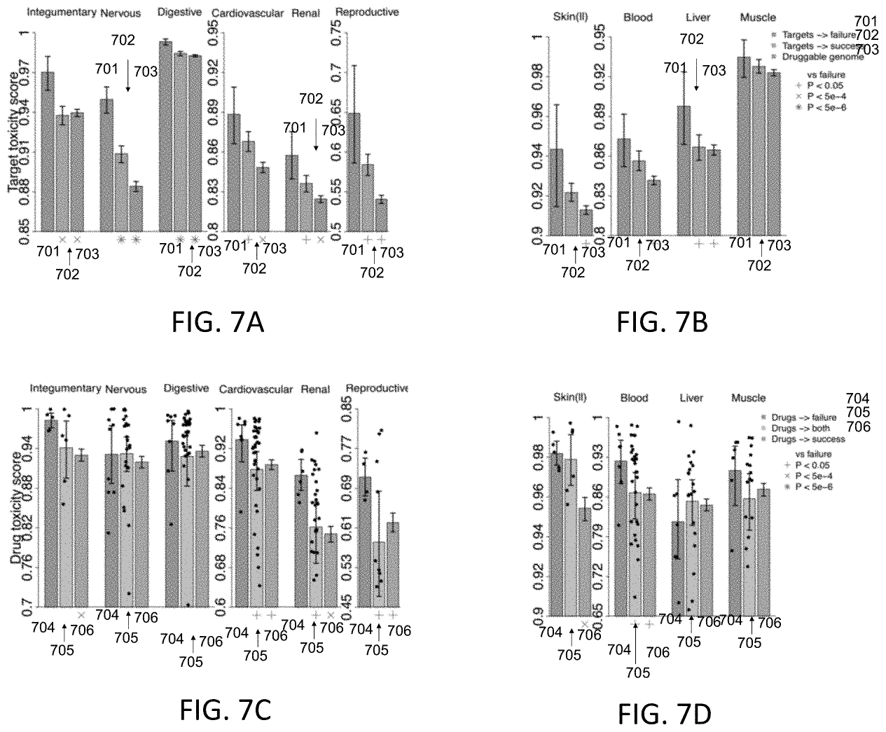

In certain embodiments, the disclosed system can construct supervised models to predict general outcomes of clinical trials. The supervised models can predict general outcomes of clinical trials based on TissueTox scores of systems/tissues can be calculated for each drug. For example, in the systems or tissues where severe side effects can be observed, the targets of trials, which can be terminated due to tissue toxicity, can have higher TissueTox scores compared to the completed targets (FIGS. 7A and 7B). FIGS. 7A-7D show comparison of TissueTox scores between targets associated with failed trials 701 and targets associated with succeeded trials 702 in 6 systems (FIG. 7A) and 4 tissues (FIG. 7B) where severe side effects were observed. TissueTox scores of all proteins in druggable genome 703 are shown as comparison. Error bar shows the 95% confidence interval calculated by bootstrap sampling. The significance levels of one-sided T test against targets associated with failed trials are shown under the x-axis. Skin(ll): skin of lower leg (sun exposed); Blood: whole blood; Muscle: skeletal muscle. In non-limiting embodiments, the disclosed system can calculate TissueTox scores of drugs by averaging the predicted scores across targets (FIGS. 7C and 7D). FIGS. 7C-7D show similar trends to FIGS. 7A-7B, except the comparison are between drugs leading to the failure of trials 704 and drugs leading the success of trials 706. Drugs leading to both outcomes 705 are shown as comparison.

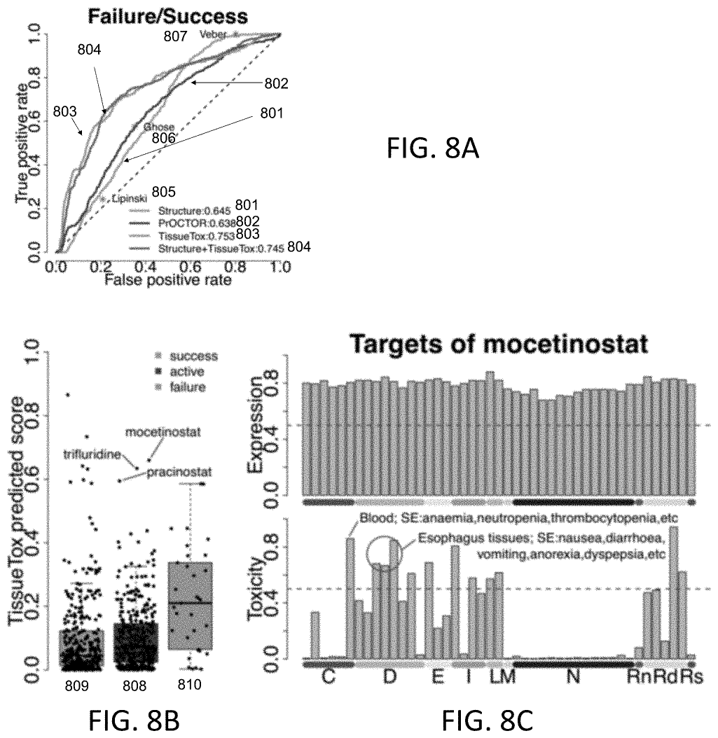

In non-limiting embodiments, chemical structure/feature information of drugs can used for the supervised models. Such chemical structure/feature information of drugs can be downloaded from a database (e.g., DrugBank). In some embodiments, as shown in FIG. 8A, binary features of drug-likeness measurements (e.g., Lipinsk's rule 805, Ghose 806, and Veber 807) can be included for the TissueTox score analysis.

FIG. 8A shows ROC curves of four classifiers predicting the outcomes of clinical trials including structural-based method 801, a previously developed method named PrOCTOR 802, TissueTox scores-based method 803, and combining structural properties with TissueTox scores 804. The structural-based method can assess a polar surface area, molecular weight, drug-likeness measurements (e.g., Lipinsk's rule 805, Ghose 806, and Veber 807). PrOCTOR can assess structure features and GTEx tissue-specific expression to predict outcomes. AUROC values are shown as legend on the bottom-right. The sensitivity (y-axis) and 1-specificity (x-axis) of three drug-likeness measurements are shown as asterisks in the plot. In some embodiments, the supervised models can be trained by using both tissue toxicity and chemical structure/feature to predict general outcomes of clinical trials.

In certain embodiments, the supervised model trained with TissueTox scores can outperform certain analyses. For example, as shown FIG. 8A shows multiple classifiers which are trained using different parameters (e.g., structure 801, proctor 802, and drug-likeness measurements 805-807). TissueTox scores can achieve an AUROC of 0.753 with a 17% increase from structure-based approach. In non-limiting embodiments, the disclosed system can integrate various data and include multiple analyses to predict success rates of clinical trials. For example, structure, proctor, TissueTox, or a combination of thereof can be assessed by the disclosed system.

In non-limiting embodiments, the disclosed system can a tissue-specific predictions. TissueTox scores can accurately capture the tissues where side effects will occur in clinical trials. For example, FIG. 8B shows that three drugs with the highest predicted probability to fail are mocetinostat, trifluridine, and pracinostat. While the targets of mocetinostat show universal high expression across normal tissues, the disclosed system can predict them with high toxicity in a subset of tissues such as blood and esophagus (FIG. 8C). FIG. 8B shows applied the TissueTox scores-based model to 356 drugs currently undergoing clinical trials 808. The predicted probability to fail are shown. The out-of-bag probability of 337 drugs leading to success 809 and 33 drugs 810 leading to failure are also shown as comparison. FIG. 8C shows the mRNA expression (upper) and predicted toxicity (lower) of mocetinostat targets across 45 GTEx tissues. Both scores can be normalized to percentiles to enable comparison across tissues. All 45 tissues are grouped by the 10 systems on x-axis. Blood and esophagus tissues are highlighted and annotated with the side effects that occurred in those tissues. These tissues can match the sites of side effects observed in the trial such as anemia, neutropenia, nausea, and diarrhea. Similar pattern can be found in targets of trifluridine and pracinostat. The disclosed subject matter also provides methods for predicting success rates of clinical trials. In certain embodiments, an exemplary method can include constructing a training set using a data source 104, calculating a performance score and a robustness score of the training set based on selected features 105, selecting a random forest model the calculated performance and robustness scores 106; and calculating a toxicity score of the pharmaceuticals by applying the random forest model to a genome which are affected by the pharmaceuticals 107. In non-limiting embodiments, the toxicity score can be validated based on clinical trial data using the pharmaceuticals 108. The clinical trial data can be previous clinical trial data and/or pending clinical trial data. In some embodiments, the accuracy of the random forest model can be further improved by dynamically adding additional clinical trial data. For example, if new trials are completed, results of the trials can be added to the training set of the disclosed system to improve the accuracy.

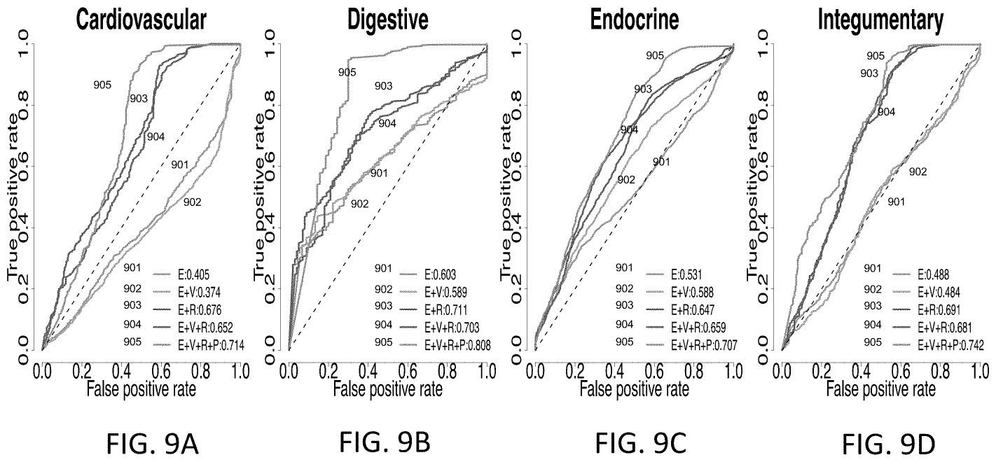

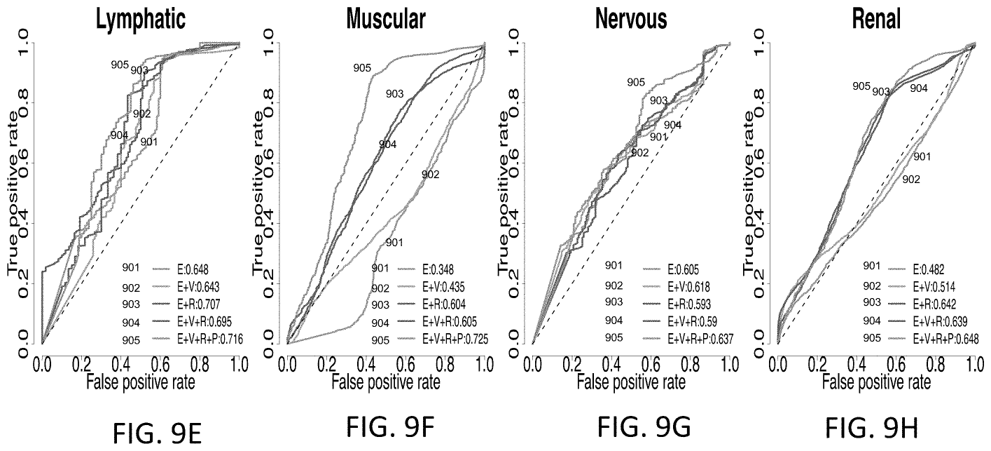

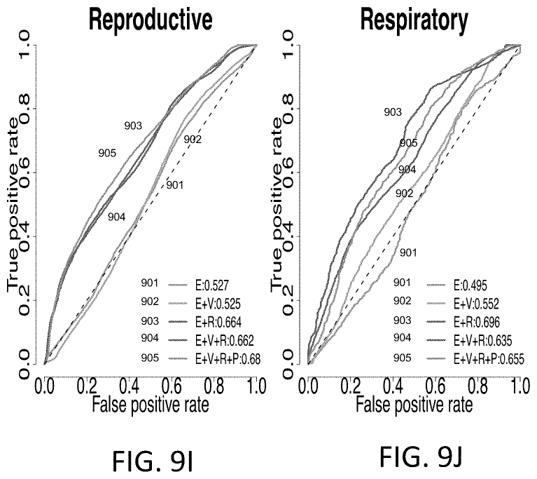

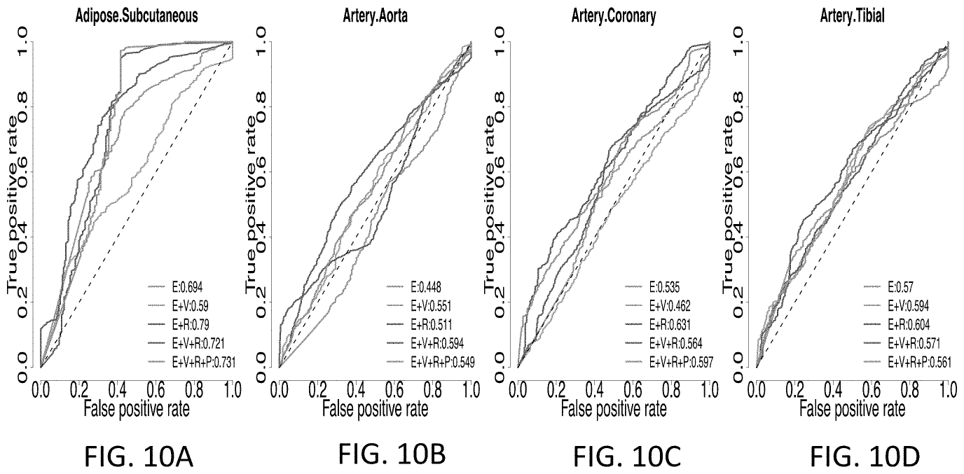

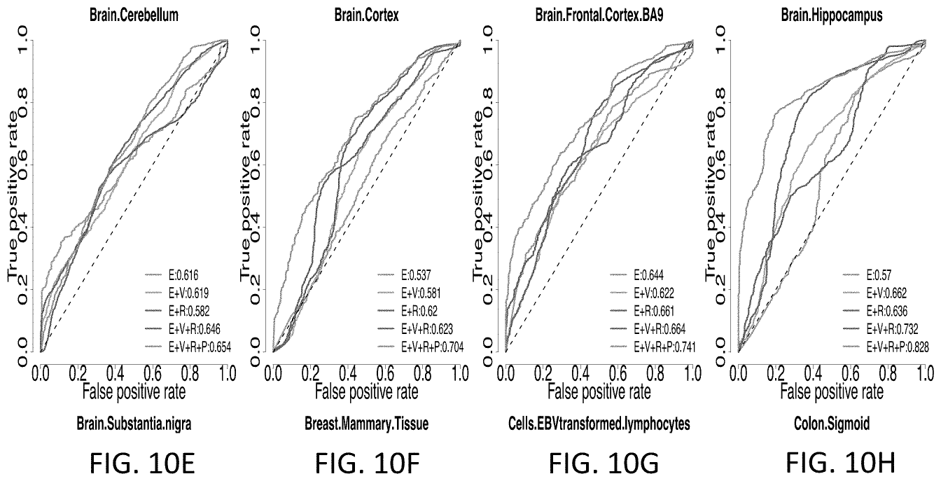

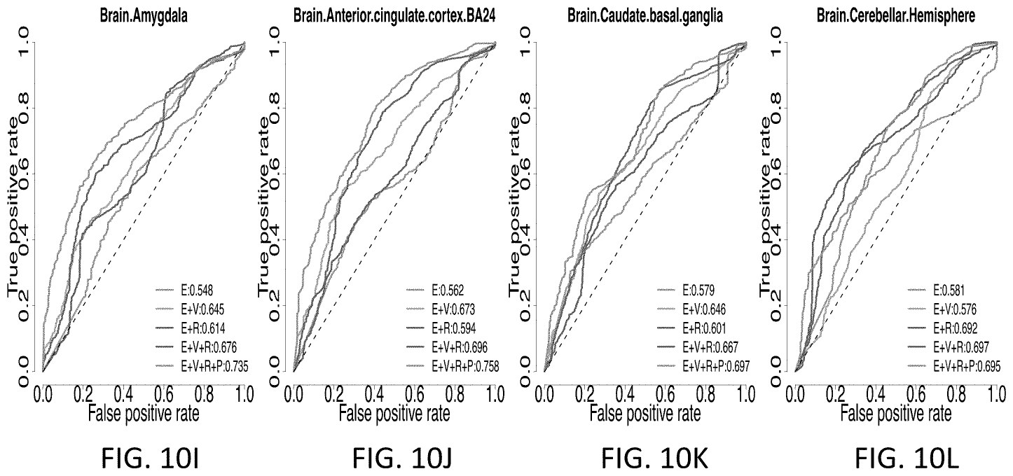

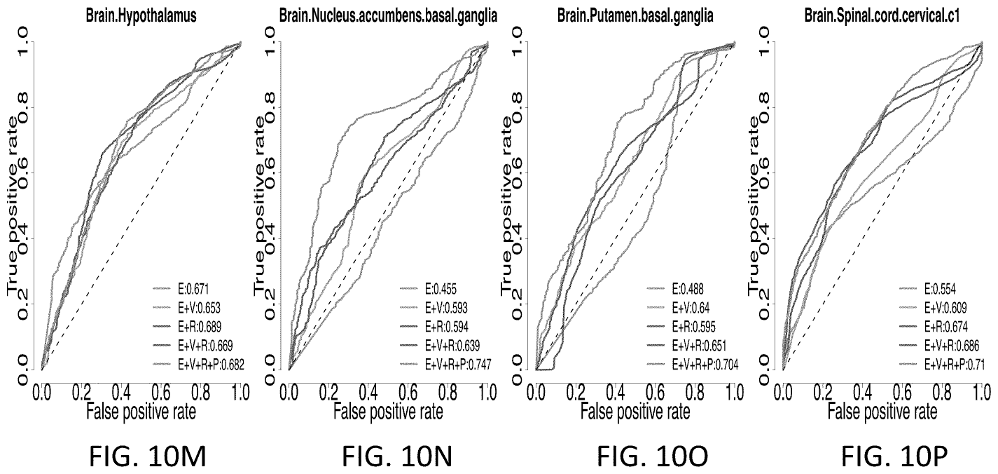

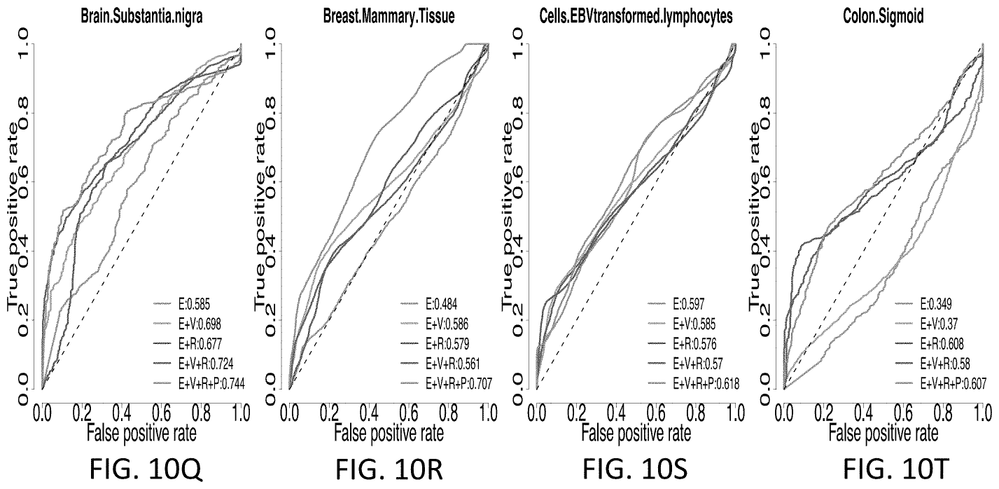

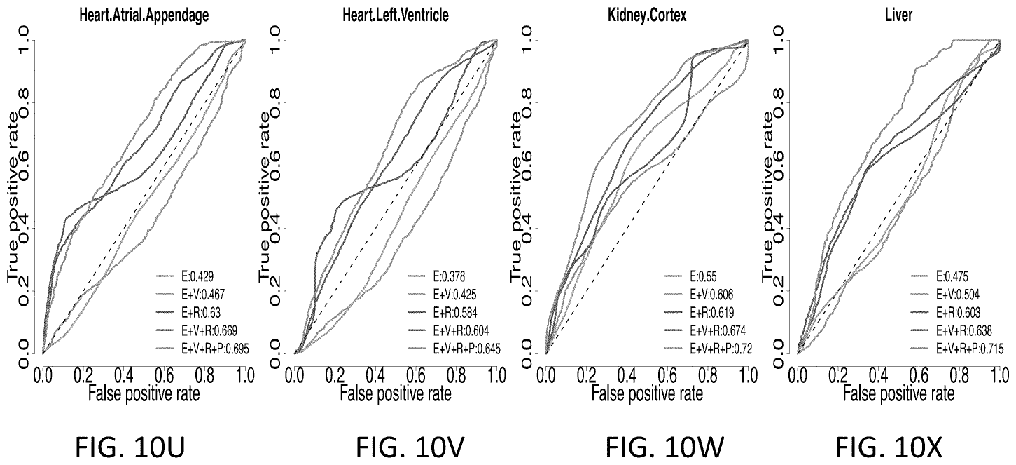

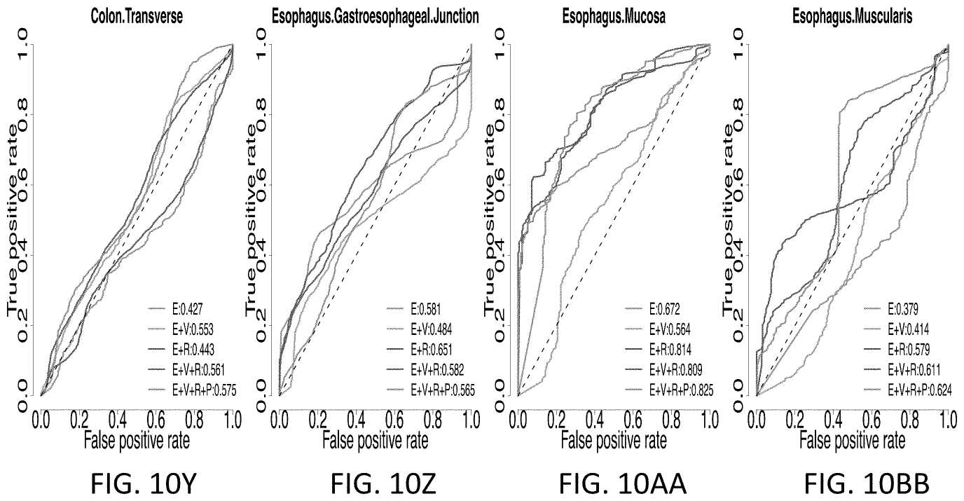

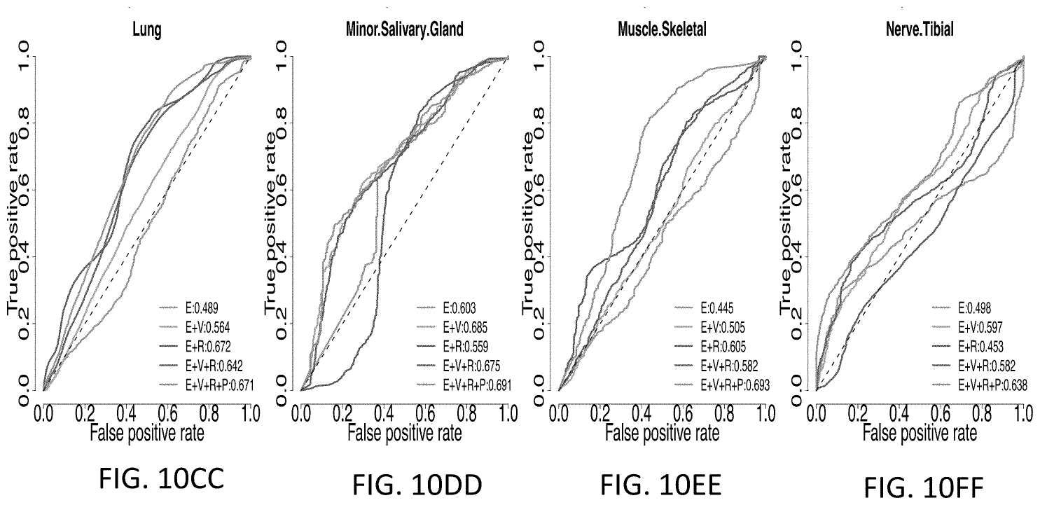

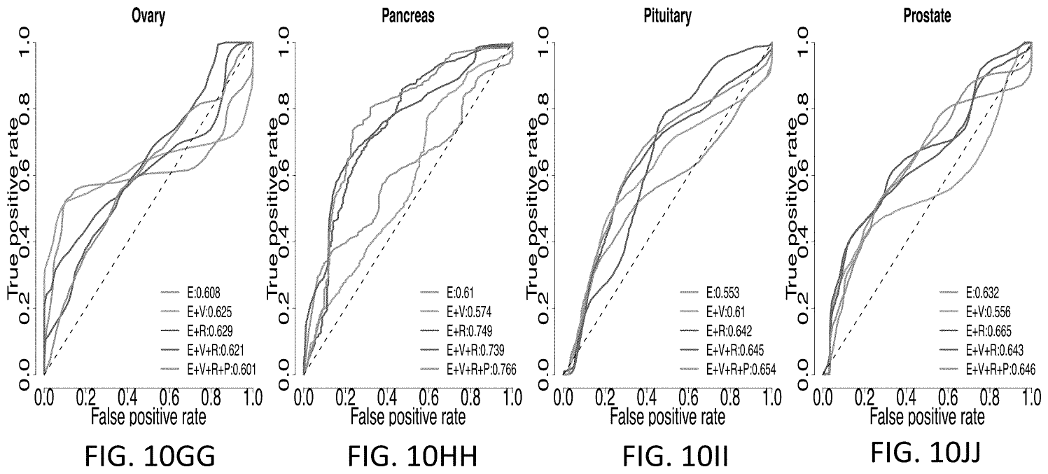

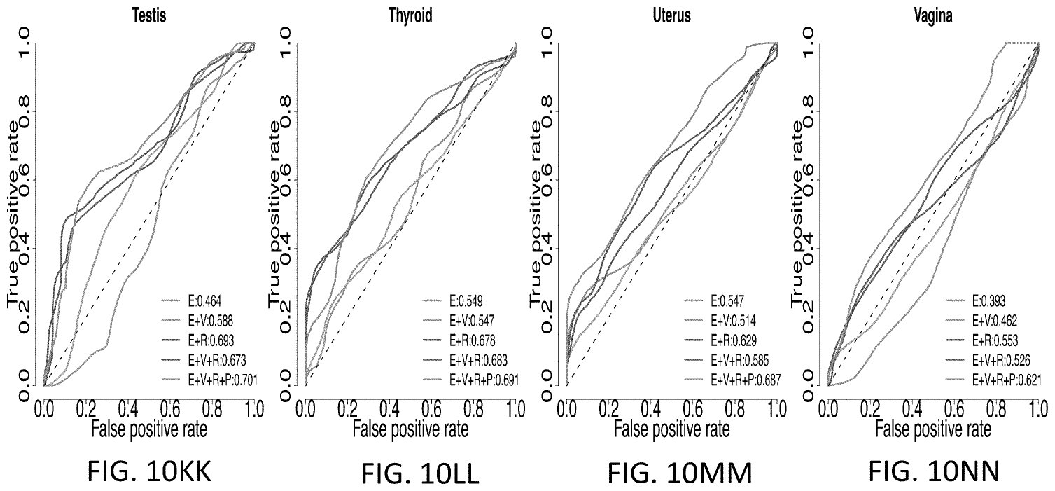

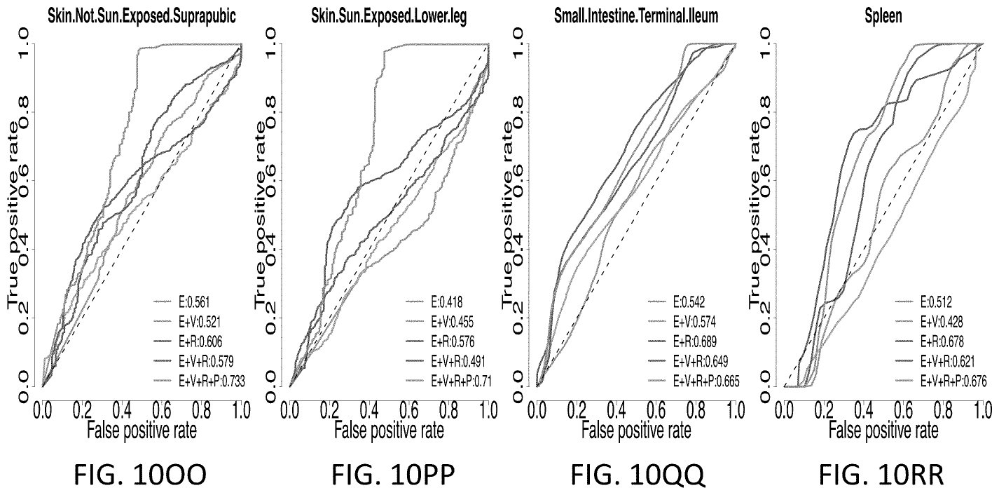

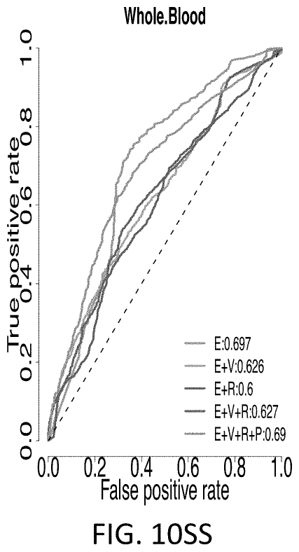

FIGS. 9-10 show performance comparison of TissueTox with other models in 10 body systems (FIGS. 9A-9J) and 45 GTEx tissues (FIGS. 10A-LOSS). The receiver operating characteristic (ROC) curves of TissueTox as well as other models can be built using one, two, or three types of features (i.e., E 901, E+V 902, E+R 903, E+V+R 904, and E+V+R+P 905). The name of each system was shown as title at the top of each plot. Abbreviation for the features: E: expression; V: Variation; R: regulatory, P: pathway.

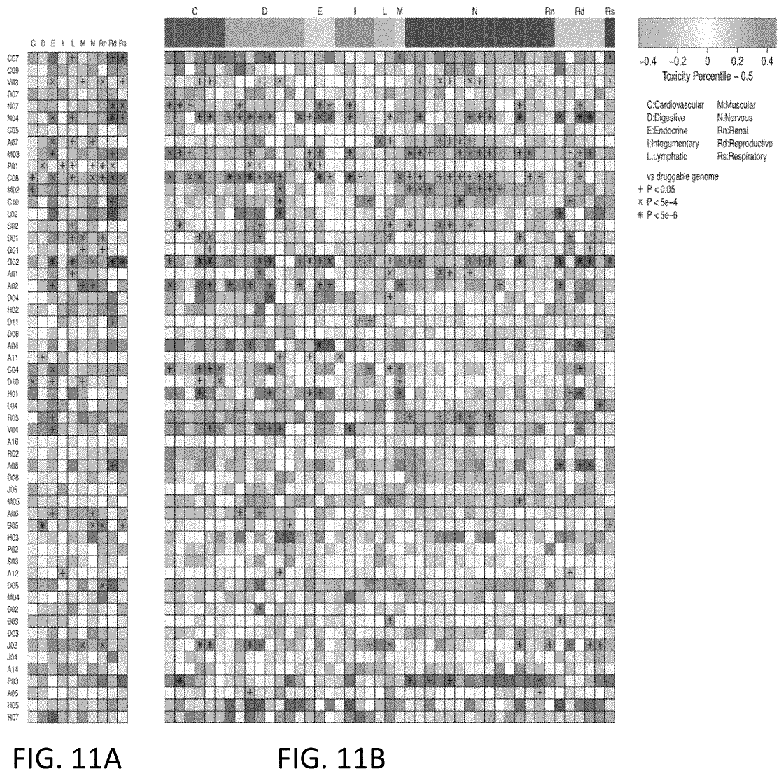

FIGS. 11A-11B show comparison of TissueTox scores across 56 ATC drug categories. The toxicity of each category can be measured by the average percentile of TissueTox scores among all 4,857 proteins. The average percentiles are shown as heatmap for 10 systems (FIG. 11A) and 45 tissues (FIG. 11B). All 45 tissues are grouped by the 10 systems on x-axis in (FIG. 11B). The significance levels oft test against all 4,857 proteins were shown in the cells with adjusted p-value less than 0.05.

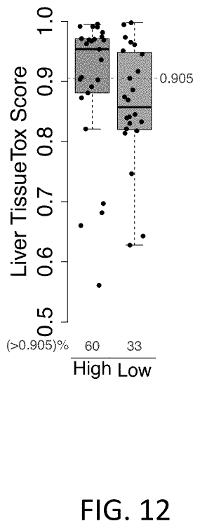

FIG. 12 shows comparison of TissueTox scores between high- and low-confidence DILI-related targets. The Liver TissueTox scores of 25 high-confidence and 24 low-confidence DILI-related targets are shown as boxplot with jitter points (37 high-confidence and 24-confidence targets are identified). The median Liver TissueTox score of all 4,857 proteins in druggable genome is 0.905, and are highlighted with dashed line in the plot. The proportion of targets with higher scores than the median are shown above the x-axis.

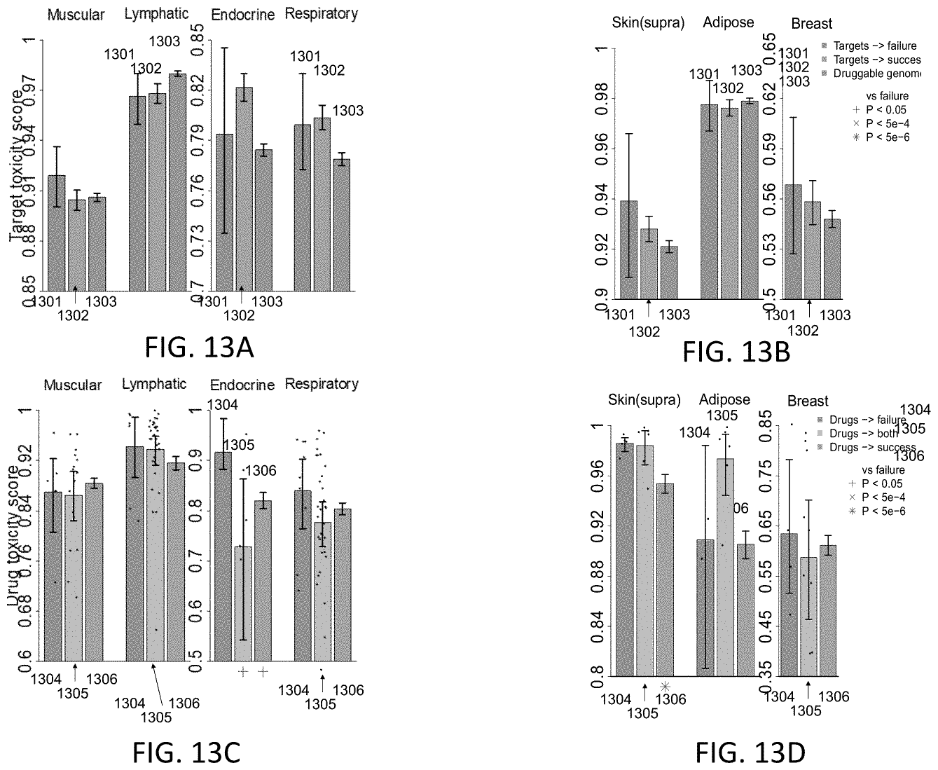

FIGS. 13A-D show comparison of TissueTox scores between failed and succeeded trials in 4 systems (FIG. 13A) and 3 tissues (FIG. 13B). FIG. 13A shows comparison of TissueTox scores between targets associated with failed trials 1301 and targets associated with succeeded trials 1302 in 4 systems (FIG. 13A) and 3 tissues (FIG. 13B) where severe side effects were observed. TissueTox scores of all proteins in druggable genome 1303 are shown as comparison. Error bar shows the 95% confidence interval calculated by bootstrap sampling. The significance levels oft test against targets associated with failed trials were shown under the x-axis. FIGS. 13C-13D showed similar trends to FIGS. 13A-13B, except the comparison was between drugs leading to the failure of trials 1304 and drugs leading the success of trials 1306. Drugs leading to both outcomes 1305 are shown as comparison.

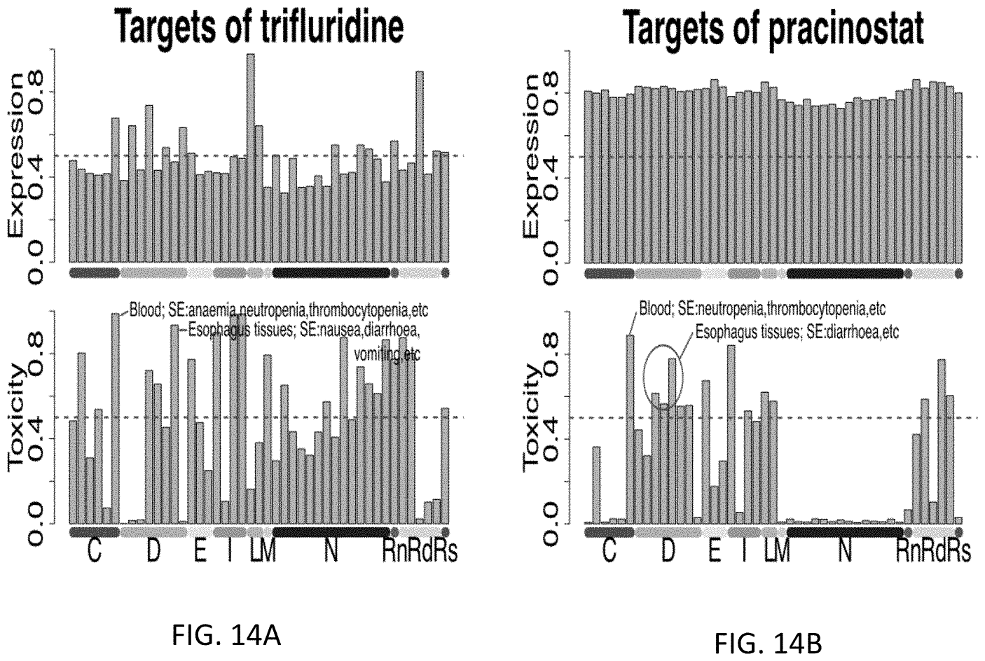

FIGS. 14A-14B shows predicted toxicity of trifluridine and pracinostat across 45 GTEx tissues. The mRNA expression (upper) and predicted toxicity (lower) of trifluridine (FIG. 14A) and pracinoistat (FIG. 14B) targets across 45 GTEx tissues. Both scores are normalized to percentiles to enable comparison across tissues. All 45 tissues are grouped by the 10 systems on x-axis. Blood and esophagus tissues are highlighted and annotated with the side effects that occur in those tissues.

The disclosed technique can be used for the assessment of toxicity in tissues or cell types where transcriptome profiling data is available. The disclosed system can predict toxicity for any protein, even those that have not yet been targeted by drugs. As tissue-specific prediction of off-targets can be provided by the disclosed technique, TissueTox can be applied to assess the off-target toxicity of drugs, which can result in more accurate prediction of outcomes for clinical trials.

### EXAMPLES

The presently disclosed subject matter will be better understood by reference to the following Examples. These Examples are provided as merely illustrative of the disclosed methods and systems, and should not be considered as a limitation in any way.

### Methods

Selection of objects: Both tissues and organ system level of tests were performed. Forty-five human tissues were selected from GTEx consortium based on the data availability, and further classified into 10 organ systems based on anatomy (FIGS. 9A-9J). The following steps were applied to build one TissueTox model for every tissue/system.

Construction of training sets: No existing resource provides standards that directly connect target proteins to tissue toxicity. The connections by integrating three existing resources, SNOMED, SIDER, and DrugBank were established. For each tissue/system, related side effect terms were extracted from SNOMED using semantic relationship of “finding_site_of”. Positive and negative control drugs of every side effect were obtained from SIDER and SIDERctrl, respectively. SIDERctrl, which use biological and chemical properties of drugs, was developed to identify negative control drugs from all the unreported drugs of each side effect. SIDERctrl can reduce the false negative rate of unreported drugs by one-third to one-half. Target proteins of each drug were obtained from DrugBank. Since the target annotations in DrugBank are mostly on-targets of drugs, the following filtering process was applied to reduce the mismatch between on-targets and off-target side effects:
1. For each drug D, the probability of causing tissue toxicity (TT) P_{D→TT} was calculated as

$${P}_{D \to TT} = \frac{N ( related SEs caused by D )}{\begin{matrix}N ( related SEs caused by D ) + \\ N ( related SEs in which D is negative control )\end{matrix}} \qquad ( 9 )$$

A threshold T_{D} was used to define tissue toxicity of drugs as

$${P}_{D \to TT} = \{ \begin{matrix}[ 0 , {T}_{D} ] & negative control \\ ( {T}_{D} , 1 - {T}_{D} ) & removed from the set \\ [ 1 - {T}_{D} , 1 ] & tissue factory\end{matrix} \qquad ( 10 )$$

2. For each target protein P, the probability of causing tissue toxicity P_{P→TT} was calculated as

$${P}_{P \to TT} = \frac{N ( drugs targeting P that cause TT )}{\begin{matrix}N ( drugs targeting P that cause TT ) + \\ N ( drugs targeting P that do not cause TT )\end{matrix}} \qquad ( 11 )$$

The same method was used to define tissue toxicity of target proteins with a threshold T_{P}.

Five different values 0, 0.1, . . . , 0.4 to T_{D} and T_{P} were applied, respectively. As a result, 25 training sets were derived for each tissue/system. Training sets with less than ten positive or negative samples were removed to prevent overfitting. The best value of T_{D} and T_{P} was selected by a process described below, which identified the training set with the least noise.

Calculation of target features: Four types of target features were incorporated in every TissueTox model: expression, variation, pathway, and regulatory.

Expression: TissueTox calculated two expression features per tissue, which indicated the absolute and differential expression of a target in the tissue, respectively. Absolute expression was measured by the percentile of RPKM value among all genes. Replicates of the same tissue were averaged. Differential expression was measured by the absolute fold change derived from DESeq analysis. For each tissue type, the control samples were generated using the following method. First, samples from other tissues of the same body system were removed due to high similarity in expression. Next, the remaining tissues were averaged across replicates then grouped by the body system. Ten bootstrap samples were drawn from each system to account for the imbalanced number of GTEx tissues from different systems. The bootstrap samples were used as control for DESeq analysis. Log transformation was applied to the original fold change value to adjust for highly skewed distributions.

Variation: TissueTox adopted two tissue-naïve variation features, Residual Variation Intolerance Score (RVIS) and Haploinsufficiency (HI) score, which measure the tolerance of a target to genetic mutations. The two features are consistent across all TissueTox models.

Pathway: TissueTox used Reactome as the data source for pathways. Two data-driven methods, GOTE and DATE, which connected G-protein coupled receptors (GPCRs) or non-GPCRs, were developed to tissue-specific functional pathways, respectively. The two methods were designed for expression datasets containing one sample per tissue. An enhanced version of the methods was introduced: MS-GOTE and MS-DATE, which can cope with multi-sample expression datasets such as GTEx. The methods to predict tissue-specific downstream pathways of targets were applied. Pathways with less than 5 or more than 100 annotated proteins were considered as incompletely or excessively annotated, thus were eliminated from the results. In addition, to reduce the redundancy among predicted pathways, the hierarchy of Reactome was used to filter out pathways that were connected to a target along with their descendants. Each predicted pathway was regarded as a binary feature in the TissueTox model, which indicated whether the pathway was connected to a target or not.

Regulatory: TissueTox calculated two regulatory features per tissue: recall and precision, which measured the efficacy of targets modifying the activity of master regulators through downstream pathways (DPs). First, ARACNe was applied to infer tissue-specific gene regulatory network from normalized mRNA expression data (RPKM) of each GTEx tissue, then VIPER was used to infer the activity of transcription factors (TFs) regulating gene expression. TFs with significant activity (P<0.05) were defined as master regulators (MRs). Recall was defined as the weighted proportion of MRs that are regulated by the DPs of a target while precision was defined as the weighted proportion of DPs that effectively regulate MRs. Specifically,

$$\begin{matrix}Recall = \frac{{Σ}_{i ∈ MRs} - log {P}_{i} * I ( i annotated in DPs )}{{Σ}_{i ∈ MRs} - log {P}_{i}} & ( 12 ) \\ Precision = \frac{{Σ}_{j ∈ DPs} \frac{- log {P}_{j}}{length ( j )} * I ( j contains MRs )}{{Σ}_{j ∈ DPs} \frac{- log {P}_{j}}{length ( j )}} & ( 13 )\end{matrix}$$

where I is the indicator function, MRs are weighted by the p-value derived from VIPER analysis Pᵢ, and DPs are weighted by the ratio of p-value derived from the pathway analysis Pⱼ versus the number of proteins in the pathway.

Training and selection of TissueTox model: Using the features above, 100 random forest classifiers with 500 trees each were built for every training set derived for a tissue/system. The parameters of random forest were set. Results were averaged over the 100 classifiers to account for the stochastic nature of random forest. The out-of-bag probability was used to evaluate the performance of each model, which was measured by the AUROC. To prevent overfitting, 10, 20, . . . , 50 percent samples or features were randomly removed from each training set and recalculated the AUROC of new models. The removal was repeated 100 times to account for the stochastic nature of sampling. Two linear regression models were fit using the normalized AUROC against the percentage of samples and features left to rebuild the model. The model robustness was measured by the absolute coefficients of two linear models: kₛₐₘₚₗₑ and k_{feature}. The performance and robustness scores were normalized across all models derived for the same tissue/system using median absolute deviation (MAD) modified Z-scores, which were then combined using Stouffer's method. Specifically

$$\begin{matrix}Modified {Z}_{i} = \frac{0.6745 ( {x}_{i} - \overline{x} )}{MAD} & ( 14 ) \\ Combined Z = \frac{{w}_{AUROC} * {Z}_{AUROC} + {w}_{{k}_{sample}} * {Z}_{{k}_{sample}} + {w}_{{k}_{feature}} * {Z}_{{k}_{feature}}}{\sqrt{{w}_{AUROC}^{2} + {w}_{{k}_{sample}}^{2} + {w}_{{k}_{feature}}^{2}}} & ( 15 )\end{matrix}$$

where w_{AUROC}, wₖ_{ₛₐₘₚₗₑ}, wₖ_{_{feature}} are the weights used to combine three measurements and were set as 1, 0.5, 0.5 to ensure that performance and robustness were equally considered in model selection. The model with the highest combined Z-score was selected for each tissue/system. The importance of each feature was measured by the increase in mean squared error (MSE) when the feature was removed from the model. The importance score was then normalized by the sum across all features in each model.

Application of TissueTox model to the human druggable genome: The human druggable genome containing 4,857 proteins were curated by integrating three databases: dGene, GtoPdb, and DrugBank. All druggable proteins were classified into seven major classes: GPCRs, nuclear hormone receptors, ion channels, transporters, catalytic receptors, enzymes, and other proteins. The selected random forest model of each tissue/system was applied to calculate the probability of causing tissue toxicity, which was defined as the TissueTox score.

Identification of toxic proteins for Gene Ontology enrichment analysis: Toxic proteins were defined as proteins with TissueTox scores higher than the median of druggable genome in all ten body systems (FIGS. 11A and 11B). Gene Ontology (GO) enrichment analysis of toxic proteins was performed using PANTHER (FIG. 12). GO terms were analyzed by three distinct categories: biological process, molecular function, and cellular component. GO terms with less than 5 or more than 100 annotated genes were eliminated from the results.

Comparison of TissueTox scores across ATC drug categories: ATC classification of drugs were obtained. The level two hierarchy (first three digits) was applied to classify drugs into 76 categories. For each target protein, the percentile of TissueTox scores was calculated among the druggable genome to enable comparison across distinct tissues or systems. The distribution of percentile scores in each ATC category was compared to the whole druggable genome using two-sided T test (FIGS. 13A-13D). Bonferroni correction was performed to adjust for multiple testing across ATC categories.

Validation of TissueTox score using clinical trials data from AACT: Curated data of clinical trials was obtained from AACT database. The “studies.txt” file was used to extract 74 trials failed for toxicity reasons and 8,419 trials as negative controls. The failed trials were identified by overall status of “terminated”, “suspended”, or “withdrawn”, along with specified toxicity or safety reasons that led to the failure. The control trials were identified by overall status of “completed”. The “interventions.txt” file was used to extract drugs administrated in each clinical trial and the “reported events.txt” file was used to extract side effects observed, along with the tissues or systems where the side effects occurred. The tissue names adopted by AACT were manually mapped to GTEx tissues. To ensure that the validation is independent of the model construction, the drugs or target proteins from the training sets of TissueTox models were removed if they appeared in the AACT dataset, then rebuilt the models with the rest of training data and regenerated TissueTox scores of all proteins in human druggable genome. TissueTox scores were compared on two levels: target proteins and drugs. TissueTox score of a drug was defined as the average scores of target proteins.

Construction of supervised models to predict general outcomes of clinical trials: Three types of features for the supervised models were calculated: chemical structure, PrOCTOR, and tissue toxicity.

Chemical structure: The structure information (sdf format) of drugs was downloaded from DrugBank. Ten chemical features were extracted from the sdf file. Three binary features of drug-likeness measurements were further included: Lipinsk's rule of five, Ghose, and Veber.

PrOCTOR: PrOCTOR is an algorithm integrated the chemical features of drugs described above with other properties of target proteins including mRNA expression from 30 GTEx tissues, degree and betweenness centrality in gene-gene interaction network, and loss frequency from ExAC database.

Tissue toxicity: TissueTox scores of 10 systems and 45 tissues were calculated for each drug in the validation set.

The performance of four supervised models predicting successes and failures of clinical trials were compared: structure-based, PrOCTOR, tissue toxicity-based, and structure combined with tissue toxicity. For each model, 100 random forest classifiers with 500 trees each were built. Results were averaged over the 100 classifiers to account for the stochastic nature of random forest. The out-of-bag probability was used to evaluate the performance of each model, which was measured by the AUROC.

MS-GOTE and MS-DATE (Approaches predicting the downstream signaling pathways of G-protein coupled receptors (GPCRs) and non-GPCRs): GOTE was developed to predict the downstream signaling pathways of GPCRs by tissue expression. MS-GOTE (MS: multiple sample) is an enhanced version from GOTE in that MS-GOTE can cope with multi-sample expression datasets, use information derived from multiple samples to call differential expressed genes (DEGs), as well as to infer the coupling between G-protein coupled receptors and transducers (G-proteins and β-arrestins).

In GOTE, gene expression across tissues, transformed the distribution of all genes into Gaussian, and identified tissue-specific DEGs were adjusted based on deviation from the mean. In MS-GOTE, DESeq was used to call DEGs as multiple samples of the same tissue are available. Bonferroni correction was used to adjust the p-value, and defined DEGs as genes with adjusted p-value less than 0.05. Pathway enrichment analysis was performed on the differentially expressed binding proteins of each transducer using Fisher's Exact Test, then transformed the p-value into Z-score. Pathways enriched by a transducer with higher correlation to the GPCR will be prioritized in the disclosed model. The Z-scores of each pathway derived from distinct transducers were combined using Stouffer's Z-score method:

$${Z}_{combine} = \frac{{Σ}_{i ∈ transducers} {w}_{i} * {Z}_{i}}{\sqrt{{Σ}_{i ∈ transducers} {w}_{i}^{2}}} \qquad ( 16 )$$

where the Z-score of each transducer was weighted by wᵢ.

In GOTE, wᵢ was defined as the expression of transducer Eᵢ. In MS-GOTE, the pearson correlation coefficient of RPKM was calculated across multiple samples Cᵢ, to measure the co-expression between the GPCR and transducer, which was used as an evidence to infer the coupling between them. Then wᵢ was defined as the product of Eᵢ and C. The combined Z-score was transformed to p-value, and pathways with p-value less than 0.05 were defined as downstream signaling pathways of the GPCR.

Similarly, MS-DATE incorporated the results of DESeq analysis into DATE, a previously developed approach connecting non-GPCRs to annotated pathways. In DATE, an expression Z-score was calculated based on central limit theorem to assess the tissue-specific expression of genes in a pathway, then connected a non-GPCR to an annotated pathway when the Z-score is greater than 1.64. In MS-DATE, the tissue-specific expression was assessed by testing whether the pathways genes are enriched among DEGs using Fisher's Exact Test and connected a non-GPCR to an annotated pathway when the p-value is less than 0.05.

The tissue toxicity-based model to 356 drugs was applied undergoing clinical trials, which were identified by overall status of “active, not recruiting”, “not yet recruiting”, or “recruiting”. The probability of failure was calculated for each drug using the random forest model.

### Results and Discussion

There was the knowledge gap between target proteins and side effects. Most of knowledge on the pharmacology of druggable proteins is in their therapeutic potential, while the relationships between these proteins and adverse side effects remains enigmatic. In addition, due to the difficulty of inferring causal relationship between targets and tissue-specific effects, there are few known examples, making it difficult to develop systematic approaches predicting tissue toxicity in general.

To address this fundamental problem, a target-based algorithmic framework, TissueTox, for the prediction of tissue toxicity was established (FIGS. 2A-2E). Using data from 548 drugs and 620 side effects in 45 human tissues and 10 body systems (FIGS. 9A-9J), a reference dataset of targets and tissue toxicity were defined. A supervised model was trained using this reference dataset for each of the 10 systems and 45 tissues. In TissueTox, four types of multi-omic features including mRNA expression, tolerance to genetic variation, interaction with cellular regulatory networks, and pharmacological pathways, were integrated. In total, an average of 284±27 training examples and 334±39 features per tissue/system were obtained. The best model for each tissue/system was selected based on a balance between performance and robustness. A significant improvement (P<5e-4) was observed in the performance after the regulatory and pathway features were added in the model (FIG. 3A). The median area under receiver operating characteristic curve (AUROC) was 0.711 (95% CI: 0.652-0.729) across the 10 systems (FIG. 3C and FIGS. 9A-9J) and 0.691 (95% CI: 0.671-0.704) across the 45 tissues (FIG. 3D and FIGS. 10A-10SS). The performance remained robust against the partial removal of features or samples, where 90% of original AUROC with 50% of the data was retained (FIG. 3B), suggesting that TissueTox models were not overfitting the training data. The predictive power of distinct features were also compared. Pathway features had the highest predictive power, accounting for 40±10% of the normalized importance among 10 systems (FIG. 4A) and 53±5% among 45 tissues (FIG. 4B). Genetic variation intolerance features showed the lowest predictive power. Expression features showed higher predictive power in systems (34±14%) compared to tissues (14±3%).

TissueTox was applied to assess the toxicity of 4,857 proteins in the human druggable genome, including 2,540 proteins that have been targeted by approved or experimental drugs, as well as 2,317 potential targets within druggable classes. This is the first tissue-specific toxicity profile of the human druggable genome. Then, the predicted TissueTox scores were compared across protein classes and observed distinct levels of toxicity as well as tissue-specificity within each class (FIG. 5). For instance, GPCRs were predicted with low toxicity in most systems except reproductive system while ion channels were predicted with high toxicity, in the nervous system due to their high expression in these tissues. NHRs show high variability of predicted toxicity across systems, ranging from low toxicity in the renal system to high toxicity in the reproductive system, while transporters and proteases average toxicity consistently across systems. It is worth noting that well-established targets of cancer therapy such as RTKs, STKs, PI3Ks, and PTEN exhibit high predicted toxicity in the digestive or integumentary system, where most side effects were observed among patients receiving the therapy. Based on the TissueTox scores, 60 proteins that consistently show high toxicity in all ten body systems were identified (FIGS. 11A-11B). Among the 60 proteins, 11 ligand-gated ion channels that are enriched in GABA-A receptor activity, chloride transmembrane transport, and 12 voltage-gated ion channels that are enriched in membrane depolarization, sodium ion transmembrane transport, as well as 6 RTKs, among which two have been targeted by existing cancer drugs: MET and PDGFRA were identified (FIG. 12).

The predicted scores of targets across ATC drug categories were also compared (FIG. 13). Targets of antiepileptics and psycholeptics show high predicted toxicity in most systems. This is likely because drugs in those categories target GABA-A receptors. Targets of drugs that treat congestion, COPD, and diabetes show low predicted toxicity (FIGS. 6A-6B and FIGS. 11A-11B). Meanwhile, the prediction recaptured the tissue-specific toxicity of several categories discovered by other tests, such as antineoplastics in integumentary system (P=4.4e-4) and antibacterials in respiratory system (P=2.4e-4). TissueTox scores can also recapture the connections between targets and drug-induced liver injury made by other tests. For instance, 37 high-confidence and 24 low-confidence proteins associated with drug-induced liver injury (DILI) were identified based on 11 curated pathological processes of DILI. The disclosed techniques showed that the high-confidence proteins are more likely to be predicted with higher TissueTox scores in liver compared to the low-confidence ones (OR=3, P=0.056; FIG. 12).

To further explore the application of TissueTox in drug development, the predicted scores was used to assess the toxicity of drugs administrated in clinical trials and connected the results to side effects and general outcomes of trials. In the systems or tissues where severe side effects were observed, the targets of trials were terminated due to tissue toxicity have significantly higher TissueTox scores compared to those trails that were completed (FIGS. 7A-7B and FIGS. 13A-13B). This result holds when the predicted scores across targets were averaged to compute tissue toxicity for drugs (FIGS. 7C-7D and FIGS. 13C-13D).

Using the TissueTox scores as features, a random forest classifier was trained predicting the results (i.e. success or toxicity failure) of clinical trials using a reference dataset that includes 33 failures and 337 successes. As comparison, certain classifiers were trained using structural properties, drug-likeness measurements, and PrOCTOR, which combined structure with target expression. TissueTox scores outperformed these approaches and achieved an AUROC of 0.753 (FIG. 8A), a 17% increase from structure-based approach. Combining structural properties did not further improve the performance of the model, suggesting that the two types of features are not complementary of one another. This model was applied to 356 drugs currently undergoing clinical trials. Three drugs with the highest predicted probability to fail are mocetinostat, trifluridine, and pracinostat (FIG. 8B). One trial using mocetinostat to treat follicular lymphoma was once put on hold due to toxicity concerns. While the targets of mocetinostat show universal high expression across normal tissues, they were predicted with high toxicity in a subset of tissues such as blood and esophagus (FIG. 8C). These tissues match the sites of side effects observed in the trial such as anaemia, neutropenia, nausea, and diarrhea. Similar pattern was also found in targets of trifluridine and pracinostat (FIGS. 14A-14B). These results support that TissueTox scores can accurately capture the tissues where side effects will occur in clinical trials.

TissueTox is a generally applicable approach for the assessment of toxicity in tissues or cell types with transcriptome profiling data available. TissueTox is able to predict toxicity for any protein, even those that have not yet been targeted by drugs. TissueTox can facilitate the generation of new genetic mechanism of toxicity, as well as improving drug safety. The approach can be further improved as the knowledge gap between target proteins and side effects is filled, providing more training data. Moreover, as tissue-specific prediction of off-targets becomes available, TissueTox can be applied to assess the off-target toxicity of drugs, which will likely result in more accurate prediction of outcomes for clinical trials.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein.

The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the methods and systems of the disclosed subject matter without departing from the spirit or scope of the disclosed subject matter. Thus, it is intended that the disclosed subject matter include modifications and variations that are within the scope of the appended claims and their equivalents.

What is claimed is:

1. A system for predicting a success rate of pharmaceuticals comprising:
one or more processors; and
one or more computer-readable non-transitory storage media coupled to one or more of the processors and comprising instructions operable when executed by one or more of the processors to cause the system to:
construct a training set using a data source;
calculate a performance score and a robustness score of the training set based on selected features;
select a random forest model based on the calculated performance and robustness scores; and
calculate a toxicity score of the pharmaceuticals by applying the random forest model to a genome which is affected by the pharmaceuticals.

2. The system of claim 1, wherein the system is further configured to validate the toxicity score based on clinical trial data using the pharmaceuticals.

3. The system of claim 1, wherein the system is further configured to improve an accuracy of the random forest model by dynamically adding additional clinical trial data.

4. The system of claim 1, wherein the target feature comprises a mRNA expression, a tolerance to genetic variation, an interaction with a cellular regulatory network, and/or a downstream pathway.

5. The system of claim 1, wherein the pharmaceuticals comprises a small molecule, a drug, a protein, a peptide, a virus, an enzyme, and/or a nucleic acid drugs.

6. The system of claim 1, wherein the performance score is calculated based on a median Area Under Receive operating characteristic curve (AUROC).

7. The system of claim 6, wherein the median AUROC score is above about 0.6.

8. The system of claim 1, wherein the robustness is calculated based on absolute coefficients of two linear models.

9. The system of claim 1, wherein a higher score of the toxicity score represents a lower success rate of the pharmaceuticals.

10. The system of claim 1, wherein the data source comprises a SNOMED, a SIDER, a DrugBank, and/or an Aggregate Analysis of Clinical Trials (AACT) database.

11. A method for predicting a success rate of pharmaceuticals comprising:
constructing a training set using a data source;
calculating a performance score and a robustness score of the training set based on selected features;
selecting a random forest model the calculated performance and robustness scores; and
calculating a toxicity score of the pharmaceuticals by applying the random forest model to a genome which is affected by the pharmaceuticals.

12. The method of claim 11, further comprising validating the toxicity score based on clinical trial data using the pharmaceuticals.

13. The method of claim 11, further comprising improving an accuracy of the random forest model by dynamically adding additional clinical trial data.

14. The method of claim 11, wherein the target feature comprises a mRNA expression, a tolerance to genetic variation, an interaction with a cellular regulatory network, and/or a downstream pathway.

15. The method of claim 11, wherein the pharmaceuticals comprises a small molecule, a drug, a protein, a peptide, a virus, an enzyme, and/or a nucleic acid drugs.

16. The method of claim 11, wherein the performance score is calculated based on a median Area Under Receive operating characteristic curve (AUROC).

17. The method of claim 16, wherein the median AUROC score is above about 0.6.

18. The method of claim 11, wherein the robustness is calculated based on absolute coefficients of two linear models.

19. The method of claim 11, wherein a higher score of the toxicity score represents a lower success rate of the pharmaceuticals.

20. The method of claim 11, wherein the data source comprises a SNOMED, a SIDER, a DrugBank, and/or an Aggregate Analysis of Clinical Trials (AACT) database.
